(19) United States
(12) Patent Application Publication
(10) Pub. No.: US 2011/0130637 A1
(43) Pub. Date: Jun. 2, 2011

(54) PROTEIN PRODUCT FOR MODIFYING CARDIOVASCULAR HEALTH

(75) Inventors: Dick Fitzgerald, Limerick (IE); Allan Struthers, Dundee (GB)

(73) Assignee: University of Dundee, Dundee (GB)

(21) Appl. No.: 12/933,533

(22) PCT Filed: Mar. 19, 2009
(86) PCT No.: PCT/EP2009/002040

§ 371 Date: Dec. 3, 2010

Related U.S. Application Data
(60) Provisional application No. 61/038,061, filed on Mar. 20, 2008.

(30) Foreign Application Priority Data
Mar. 20, 2008 (IE) S2008/0210

(51) Int. Cl. A61B 5/145; A61K 38/02; A61P 9/00; A61P 9/12; A61P 3/10; A61P 3/00

(52) U.S. Cl. 600/309; 514/21.92; 514/6.9; 514/15.7; 514/7.4

(57) ABSTRACT

A composition for use in the treatment or prophylaxis of conditions mediated by endothelial function in mammals is provided. The composition includes a milk protein hydrolysate generated by treating milk or milk whey protein with a proteolytic enzyme having subtilisin or subtilisin-like activity and/or glutamyl endopeptidase or glutamyl endopeptidase-like activity. The composition is useful in the management of vascular conditions.

The invention relates to a method for beneficially modifying endothelial function. The invention also relates to a method for the preparation of a composition suitable for preventing, treating and/or alleviating endothelial disorders associated with vascular disease.

An estimated 16.7 million people die every year (almost one-third of global deaths) from vascular and cardiovascular diseases (herein collectively referred to as VD/CVD's), which include myocardial infarctions, coronary artery disease, cerebral vascular disease (strokes), peripheral arterial diseases and other heart conditions. Furthermore, about 20 million people survive myocardial infarctions and strokes annually and many of these require continuing costly clinical care. By 2010 it is estimated that CVD will be the leading cause of death in developing countries (WHO 2003—Global strategy on diet, physical activity and health.

Endothelial cells are found in the interior surface of blood vessels. They therefore play a crucial role in the health and integrity of all tissues since a network of capillaries serves every tissue in the body.

The endothelium itself represents a monolayer of endothelial cells which line the entire circulatory system, for example, blood vessels, cardiac and lymphatic tissue. Endothelial cells act as selective filters by controlling the passage of different substances across their cell membranes. The permeability of endothelial cells is organ specific, for example, some are highly permeable such as those found in the renal glomerulus while others are highly impermeable such as those found in the blood-brain barrier. In terms of vascular biology, endothelial cells are involved in processes such as vasodilation and vasoconstriction, blood clotting, angiogenesis and inflammatory responses. Endothelial cells are also involved in a range of interactions with other cells. Leukocytes and molecules secreted by endothelial cells, for example, are involved in modulating inflammation and blood clotting.

The importance of endothelial function can be exemplified by its major role in the vascular system. Vascular endothelial cells play a central role in maintaining cardiovascular health through their ability to promote vasodilation, fibrinolysis and antiaggregation. Endothelial dysfunction occurs when the endothelium loses its ability to promote vasodilation, fibrinolysis and antiaggregation. Therefore, any condition or risk factor, which leads to endothelial dysfunction, can have negative consequences for cardiovascular homeostasis in mammalian health.

In addition to acting as a physical barrier between blood vessel walls and the lumen, endothelial cells secrete an array of mediators that can alternatively induce vasoconstriction, for example, endothelin-1 and thromboxane A2, or vasodilation, for example, nitric oxide (NO), prostacyclin and endothelium-derived hyperpolarizing factor (EDHF). These mediators function via a range of mechanisms to modify cardiovascular homeostasis. The production of NO, for instance, has in itself a range of important consequences for the vascular system. Primarily, NO maintains basal tone by relaxing vascular smooth muscle cells, however, it also inhibits platelet activation, secretion, adhesion and aggregation in addition to promoting platelet disaggregation. Furthermore, endothelial cell-derived NO inhibits leukocyte adhesion to the endothelium and inhibits smooth muscle migration and proliferation. Therefore, NO is a potent inhibitor of events which ultimately lead to neointimal proliferation and atherosclerosis (Anderson, 2003).

Oxidation reactions are involved in the events which lead to atherogenesis and associated endothelial dysfunction. Oxygen-derived free radical (or Reactive Oxygen Species (ROS)) levels and nicotinamide adenine dinucleotide dependent oxidase activity have been correlated with atherosclerotic risk and endothelial dysfunction (Cai et al., 2003). Several pathological conditions including hypercholesterolemia and hypertension increase the production of ROS in the vascular wall. Oxidative stress, associated with increased levels of ROS, leads to NO destruction and consequently to endothelial dysfunction. The general significance of endothelial function is exemplified by the fact that effective endothelial function in both coronary and peripheral vessels has important consequences for a multitude of physiological events. It can be seen from the literature that conditions such as CVD (Cooper et al., 2002), are associated with oxidative stress, which may in turn be linked with endothelial dysfunction.

Current therapies aimed at improving endothelial function are targeted at enhancing NO levels in the vasculature include the administration of L-arginine which acts as a substrate for endothelial nitric oxide synthase (eNOS) or compounds such as nitroglycerine or sodium nitroprusside which are involved in the release of NO. Some undesirable side-effects may be associated with these treatments. Other options for endothelial cell function improvement include gene therapy. However, some difficulties with targeted delivery and long-term biosafety still remain unanswered with respect to these gene-based therapies.

Research over the last 25 years has begun to identify and focus on food and its constituents as a source of safe, biologically active (bioactive) components having or likely to have the ability to positively influence the prevention, amelioration, day-to-day consequences or progression of various human health/medical or animal health/veterinary conditions, and/or meet the health enhancement/lifestyle desires and expectations of various consumer groups. In recent years, for instance, the use of milk components as ingredients in functional foods or nutraceuticals or use as functional foods or nutraceuticals in their own right has been gaining increasing worldwide scientific credibility. Furthermore, the increasing awareness by consumers of the link between diet and health/wellness is globally raising the demand for these product categories.

The terms functional foods and nutraceuticals generally refer to foods or food ingredients, which impart beneficial health effects beyond basic nutrition. The newer term “lifestyle foods” is applied to foods which are becoming associated by the consumer with benefits such as: general wellness, energy, alertness, weight management, physical appearance, emotional wellbeing, longevity, etc. The growing consumer interest in these emerging food categories is leading to an increase in self-medication, which is being driven mainly by the desire to avoid the undesirable side-effects associated with the use of synthetic drugs and to stem the increasing cost burden associated with conventional drug therapies.

Bioactive peptides encrypted within the primary structures of milk proteins may be released during food processing and/or during gastrointestinal transit. In many instances selection of peptide-based functional food ingredients has exploited the concept of using simulated gastrointestinal digestion (SGID) as a means of determining in vitro if an hydrolysate having a potential physiological function might survive gastrointestinal digestion. Furthermore, in many instances the selection of peptide-based ingredients may, in part, be determined by the molecular mass distribution of the peptides therein. In this regard, it has been shown, for example, that many potent ACE inhibitory peptides are di- and tripeptide sequences (FitzGerald and Meisel, 2003). If a particular bioactivity survives gastrointestinal transit it will have passed the first hurdle in reaching its target organ and ultimately having a physiological effect. Furthermore, it is well recognised that small peptides are more readily absorbed than large peptide fragments or indeed free amino acids. Therefore, it is generally recognised that short peptide fragments are more likely to have beneficial physiological effects particularly if they are to be consumed via the oral ingestion route.

One approach to assessing the ability of any bioactive peptide preparation or protein hydrolysate to have a beneficial physiological effect is to use appropriate surrogate biomarkers. In the case of VD/CVD for example, blood pressure or endothelial function measurement may be beneficially used. It is now well accepted that one of the best ways of assessing the likelihood of, for example, atherosclerosis developing or progressing, and then perhaps helping prevent or treat it, is to measure vascular endothelial function.

Endothelial dysfunction is classically associated with vascular diseases. Endothelial dysfunction has therefore been implicated in diseases and conditions such as hypertension (Olsen et al., 2001), atherosclerosis (Suwaidi et al., 2000), hyperlipidemia (Ferrario and Strawn, 2002) and heart failure (Farre and Casado, 2001). In the Western world, hypertension and hypercholesterolemia are two major risk factors that can lead to vascular disease such as atherosclerosis. Subsequently, atherosclerosis may result in a number of severe VD/CVDs, such as chronic heart failure, coronary artery disease, myocardial ischemia, myocardial infarctions, cerebrovascular accidents (CVAs), transient ischaemic attacks and peripheral arterial disease leading to intermittent claudication and limb amputation.

Measurement of vascular endothelial function is currently regarded by many experts in the VD/CVD field as an excellent surrogate biomarker, if targeted as part of an overall programme, in the prevention and/or treatment of CVD. The recent review articles by Widlansky et al., (2003) and Cohn et al., (2004) clearly outline the reasons why this measurement (endothelial dysfunction) is currently regarded as the best possible surrogate biomarker for future cardiovascular events. Cohn et al., (2004) also stated that “endothelial dysfunction” may be considered a target for cardiovascular therapy in which reversibility of dysfunction will be indicative of “improvement in risk”.

To date, measurement of endothelial dysfunction has outperformed other surrogate marker approaches, such as intima-media thickness (IMT) measurements, in predicting which therapies will prevent CVD events (Chan et al., 2003). Two large and compelling bodies of evidence exist which support the finding that endothelial dysfunction is strongly linked to future CVD events. Firstly, at least eight different studies currently show that patients with endothelial dysfunction have a greatly increased (up to nine-fold higher) incidence of cardiovascular events. Furthermore, at least four of these studies quantified endothelial function in the brachial artery (Schachinger et al., 2000; Suwaidi et al., 2000; Heitzer et al., 2001; Perticone et al., 2001; Modena et al., 2002; Gokce et al., 2003; Targonski et al., 2003). Secondly, seven different treatment examples exist where treatment induced changes in endothelial function are paralleled by treatment-induced changes in cardiovascular events. It has been shown that both endothelial dysfunction and cardiovascular events are reduced by treatments with aspirin, ACE inhibitors, spironolactone, statins and with angiotensin blockers (Farquharson & Struthers 2000; O'Driscoll et al., 1997). Many of these studies used the invasive brachial artery technique to measure endothelial function. Finally, it has been shown that after treatment to achieve the same risk factor levels, those patients with persistent endothelial dysfunction subsequently had seven times the number of CVD events compared to those with improved endothelial function (Modena et al., 2002). The only example(s) where a definite discrepancy occurred are the trials involving hormone replacement therapy. However, a score of 7 versus 1 in favour of studies in treatment induced changes in endothelial function, predicting treatment induced changes in cardiovascular events, is recognised as an excellent score for any predictive test in clinical medicine. Table 1, adapted from Widlansky et al., (2003), clearly demonstrates the consistent association observed between the effects of different interventions on endothelial function as linked to cardiovascular events. (The data used to generate Table 1 was referenced with 27 articles, in 17 of which the endothelial function measurement was carried out as part of an invasive arterial study).

TABLE 1

| | | | References quoted |
|---|---|---|---|
| | Effect on | | by Widlansky |
| | Endothelial | Effect on | Invasive vs non- |
| Intervention | Function | CVD Events | invasive studies |
| Lipid lowering | + | + | 5 vs 0 |
| Smoking cessation | + | + | 0 vs 1 |
| Exercise | + | + | 4 vs 2 |
| ACE Inhibitors | + | + | 2 vs 1 |
| Angiotensin Blockers | + | + | 1 vs 1 |
| N-3 fatty acids | + | + | 0 vs 1 |
| Glycaemic control in | + | + | 1 vs 0 |
| diabetes | | | |
| Vitamin E | ± | − | 4 vs 1 |
| Hormone replacement | ± | − | 0 vs 3 |
| | | | 17 vs 10 |

Vita and Keaney, (2002), as part of an editorial in Circulation, designated endothelial function as “a barometer” of vascular health representing an orchestrated response to all the processes that contribute to atherosclerosis development and progression. Vogel (2003) further emphasised this point when describing endothelial function as a gauge of both cumulative risk factor burden and genetic susceptibility, which recognises that individuals have different endothelial responses to the same risk factor burden. This recognises that these different endothelial responses are due to genetic susceptibility and to other as yet undiscovered risk factors. The real strength in endothelial function measurement is that it quantifies the end product of all the genes and environmental risk factors involved on the key target organ, the vasculature, without having to wait decades before all potential risk factors are identified. This is especially important since currently identified risk factors are thought by many to only explain approximately 50% of CVD events. Given the evidence and authoritative reviews available in the scientific literature, it is now therefore well accepted that promising therapeutic candidates for VD/CVD and related peripheral vascular conditions can be selected on the basis of how they may influence endothelial function. Vascular endothelial function is therefore well accepted and recognised as a very important surrogate marker in the field of identifying CVD therapy and as an excellent predictor of future myocardial infarctions and strokes.

It is therefore clear that a means to beneficially modify endothelial function would have valuable therapeutic and/or preventative potential in managing VD/CVD.

### STATEMENTS OF INVENTION

According to the invention, there is provided a composition for use in the treatment or prophylaxis of conditions mediated by endothelial function in mammals, comprising a milk protein hydrolysate prepared by treating milk or milk whey protein with a food grade proteolytic enzyme having subtilisin or subtilisin-like and/or glutamyl endopeptidase or glutamyl endopeptidase-like activity. The proteolytic enzyme may be a proteolytic enzyme or enzymes derived from Bacillus species. In one arrangement, the proteolytic enzyme is derived from Bacillus licheniformis. In a preferred arrangement, the enzyme comprises Alcalase™, an enzyme preparation available from Novo Nordisk A/S. The endothelial function may be endothelial dependent relaxation function. The endothelial dependent relaxation function may be endothelial dependent vasodilatation.

In one embodiment the endothelial dependent relaxation activity is stimulated by greater than 10%, greater than 20%, typically greater than 30%.

In one embodiment the protein is a whey derived protein.

The protein hydrolysate may be fractionated.

In one embodiment the hydrolysate contains greater than 60% of peptide material having a molecular weight of less than 2 kDa, greater than 70% of peptide material may have a molecular weight of less than 2 kDa, preferably greater than 80% of peptide material having a molecular weight of less than 2 kDa.

In one embodiment greater than 55% of the peptide material has a molecular weight of less than 1 kDa, greater than 65% of the peptide material may have a molecular weight of less than 1 kDa, greater than 40% of the peptide material may have a molecular weight of less than 500 Daltons.

In one embodiment the whey protein hydrolysate has a degree of hydrolysis of greater than 10%, preferably from about 15% to about 25%. The degree of hydrolysis may be approximately 19%.

Vascular diseases, disorders or conditions in mammals may be treated by the composition of the invention. In particular, it is useful in the treatment, prophylaxis or management of vascular conditions such as coronary artery disease, cerebral vascular disease and peripheral vascular disease. Also treatable by the composition of the invention are disorders which are known risk factors for the development of vascular disease, including pre-hypertension, hypertension, hypercholesterolemia and diabetes mellitus.

In another aspect the invention provides use of a whey protein hydrolysate for the prophylaxis and/or treatment of any one or more of vascular conditions such as coronary artery disease, cerebral vascular disease and peripheral vascular disease, as well as conditions which are risk factors for these, including pre-hypertension, hypertension, hypercholesterolemia or diabetes mellitus.

The whey protein hydrolysate may be present in the composition of the invention at between 5 g and 18 g.

Typically, the whey protein hydrolysate is present in the composition at approximately 14 g.

In one embodiment the composition includes one or more ingestible carrier such as a food-grade digestible carrier or a pharmaceutically acceptable carrier in the form of a liquid, a capsule, tablet or powder.

Whilst a composition for oral administration is preferred, other dosage forms for alternative routes of administration such as systemic or topical delivery are contemplated to be within the scope of the invention.

The composition may include an adjuvant.

Ideally, the composition is provided in a delivery system which delivers a desirable daily dosage amount of the beneficial-hydrolysate.

The composition may further include a drug entity. The drug entity may be selected from one or more of an antihyperlipoproteinemic agent, an antiatherosclerotic agent, an antithrombotic/fibrinolytic agent, a blood anticoagulant, an antiarrhythmic agent, an antihypertensive agent, a vasopressor, a treatment agent for congestive heart failure, an antianginal agent, an antibacterial agent, and an activator of endothelial NO synthase.

In one embodiment the drug entity is selected from any one or more of aspirin, statin, ACE inhibitor, diuretic, beta blocker, folic acid, vasodilator such as calcium antagonists and nitrates, fish oil or angiotensin blocking drugs.

The composition may include a biological compound.

The invention also provides a whey protein hydrolysate/ingredient comprising greater than 60% of peptide material having a molecular weight of less than 2 kDa. Greater than 70% of the peptide material may have a molecular weight of less than 2 kDa, preferably greater than 80% of peptide material having a molecular weight of less than 2 kDa.

In one embodiment greater than 40% of the peptide material has a molecular weight of less 1 kDa, preferably greater than 40% of the peptide material has a molecular weight of less than 500 Daltons.

In one embodiment the whey protein hydrolysate has greater than 95% solubility between pH 2.0 to 8.0, preferably greater than 80% solubility between pH 2.0 to 8.0.

The whey protein hydrolysate may have a foam stability of less than 10% after 15 minutes standing following foam formation.

The whey protein hydrolysate may have a foam stability of less than 5% after 15 minutes standing following foam formation.

In another aspect the invention provides a process for the preparation of a milk protein hydrolysate especially for use in stimulating endothelial function comprising the steps of:
optionally reconstituting or hydrating a milk protein;
hydrolysing a milk protein with a food grade proteolytic enzyme having subtilisin or subtilisin-like and/or glutamyl endopeptidase or glutamyl endopeptidase-like activity; and
fractionating the hydrolysed milk protein product.

The proteolytic enzyme may be derived from Bacillus species, for example from Bacillus licheniformis. Such activity is provided by the commercially available enzyme preparation Alcalase™ (Novo Nordisk A/S).

Also provided is a process for the preparation of a milk protein hydrolysate comprising hydrolysing milk with a food grade proteolytic enzyme having subtilisin and/or glutamyl endopeptidase activity. The enzyme may comprise a proteolytic enzyme from Bacillus species. Ideally the enzyme Alcalase™ from Bacillus licheniformis is selected. The process may include pre-treating milk to separate a fraction comprising whey protein and optionally concentrating the whey protein fraction.

The hydrolysate is ideally treated to separate from it a fraction comprising species of 5 kDa and lower.

Conveniently the hydrolysis is carried out at a temperature of between 30° C. and 70° C., more conveniently at a temperature of between 40° C. and 60° C., and most conveniently at a temperature around 50° C. and conveniently at a pH of between 4 and 9, more conveniently at a pH of between 6 and 8 and most conveniently at around neutral pH.

The milk protein hydrolysate may be fractionated by any one of membrane processing steps particularly ultrafiltration or chromatographic separation.

The milk protein is ideally whey derived protein.

In another aspect, the invention provides a method for preventing or treating vascular or cardiovascular conditions, disorders or diseases in mammals comprising administering an effective dose of the composition described above. The method may have applications in preventing or treating coronary artery disease, cerebral vascular disease including stroke or peripheral arterial disease, or conditions which represent risk factors for vascular disease, including pre-hypertension, hypertension, hypercholesterolemia, and diabetes mellitus.

Also provided is a method for monitoring cardiovascular therapy comprising the determination of vascular endothelial function before and after administration of the composition of the invention.

The invention provides means for improving the prevention and treatment of vascular and cardiovascular diseases and conditions of the vasculature in mammals by providing a composition and method for the improvement of vascular endothelial function. Such diseases and conditions include coronary artery disease, cerebral vascular disease (stroke) and peripheral arterial disease. The composition and method of the invention also have utility in addressing diseases and conditions which are known to be risk factors for the development of vascular disease, such as pre-hypertension, hypertension, hypercholesterolemia and diabetes mellitus.

Such an improvement in endothelial function may help to prevent the initiation or progression of VD/CVD.

The invention provides a composition capable of effectively improving vascular endothelial function.

The invention also provides a method for preparing composition(s) capable of effectively improving vascular endothelial function, specifically by the enzymatic hydrolysis of milk or whey proteins and more specifically by preparing a fractionated low molecular weight hydrolysate from a whole milk hydrolysate.

Since smoking is known to be one of the risk factors for the development of coronary artery disease, cerebral vascular disease (stroke) and peripheral arterial disease, the compositions and methods of the invention are useful in the prophylaxis or treatment of such disease in smokers.

As used herein, the word “milk” refers principally to dairy milk from farmed domesticated mammals including bovines, ovines, porcines, caprines, buffalo etc. In particular, the milk is produced by cows.

### BRIEF DESCRIPTION OF THE DRAWINGS

The invention will be more clearly understood from the following description of some embodiments thereof, given by way of example only, with reference to the accompanying drawings, in which:

FIG. 1 is a graph showing the degree of hydrolysis (DH %) versus hydrolysis time (min) for the hydrolysis of WPC75 with Alcalase™ 2.4 L;

FIG. 2 are graphs showing reversed-phase HPLC profiles of WPC 75 Alcalase™ hydrolysate generated at semi-pilot (1000 L) scale (a) before and (b) after simulated gastrointestinal digestion (SGID);

FIG. 3 are graphs showing reversed-phase HPLC profiles of WPC 75 Alcalase™ 5 kDa permeate generated at semi-pilot (1000 L) scale (a) before and (b) after simulated gastrointestinal digestion;

FIG. 4 is a graph showing changes in forearm blood flow (FBF) responses to acetylcholine after oral ingestion of placebo and WPC 75 Alcalase™ 5 kDa permeate in pre-hypertensive human volunteers *p<0.01, **p=0.001.

FIG. 5 is a graph showing changes in systolic blood pressure over four weeks between placebo (unhydrolysed/intact WPC) and WPC75 Alcalase™ 5 kDa permeate.

FIG. 6 is a graph showing nitrogen solubility of WPC 75 Alcalase™ hydrolysate (AC9) and WPC 75 Alcalase™ 5 kDa permeate and retentate as a function of pH; and

FIG. 7 are graphs showing foam expansion (a) and foam stability (b) properties of whey protein concentrate Alcalase™ hydrolysate and associated permeate and retentate fractions as a function of pH.

### DETAILED DESCRIPTION

We have found that milk protein hydrolysates, in particular a fractionated whey protein hydrolysate can bring about significant improvements in vascular endothelial function.

The improvement in vascular endothelial function is achieved using natural products derived from milk proteins rather than synthetic drugs. The public, provided they are educated in their use, would much prefer to take natural products instead of synthetic drugs. Synthetic drugs are more likely to produce adverse side effects, for example, those associated with synthetic ACE inhibitors (FitzGerald et al., 2004).

The invention provides a composition and a method of helping to improve vascular health, but does not alter the conventional risk factors of high blood pressure and high cholesterol. It is anticipated by using the products of the invention that the incidence of vascular disease including myocardial infarctions and strokes may be reduced because they improve endothelial function by a margin of approximately 34% based on our results to date. This could translate into a large reduction in myocardial infarctions and strokes (Widlansky et al., 2003; Cohn et al., 2004).

A composition, comprising the milk protein hydrolysates of the invention, which beneficially modifies endothelial function, may be used for preventing, treating or beneficially managing vascular and cardiovascular diseases.

In particular, the milk protein hydrolysates may be used for preventing or treating other related conditions such as atherosclerosis.

We found that enzymatically hydrolysed milk proteins, particularly whey protein hydrolysate (prepared using a food grade, proteolytic enzyme) constitute a product capable of significantly improving vascular endothelial function in mammals, in particular in humans.

We have shown that a milk protein hydrolysate, and particularly a whey proteinhydrolysate, significantly improves a surrogate biomarker for vascular disease and cardiovascular events. Since this biomarker, endothelial function, is capable of predicting future vascular and cardiovascular disease and events, such as myocardial infarctions and strokes and also other related diseases and conditions of the vasculature, and providing additional approaches to engaging in therapy for these diseases, the invention has particular significance in the whole field of prevention and treatment of such conditions. VD/CVD's, for instance, is a very complex multi-factorial disease state, therefore any method to eliminate or minimise its negative impact would be very beneficial.

Enzyme systems useful for the purpose of generating the hydrolysate of the present invention include subtilisin and/or glutamyl endopeptidase proteolytic/peptideolytic activities. Suitable sources for such enzymes or enzyme combinations include Bacillus species. One such suitable source comprises Bacillus licheniformis. Alcalase™ is an enzyme preparation from Bacillus licheniformis obtainable commercially from Novo Nordisk A/S. It contains endoproteinase (Subtilisin Carlsberg (EC 3.4.21.62)) along with endopeptidase (mainly glutamyl specific, glutamyl endopeptidase (GE)) activity (Spellman et al., 2005). Subtilisin and subtilisin-like activities are relatively non-specific proteinases, but preferentially cleave peptide bonds after large non-β-branched hydrophobic residues.

Studies on Alcalase™ specificity show that a significant number of peptides present in Alcalase™ digests of whey protein isolate had a glutamic acid residue at the C-terminus. This could be explained by the presence of a glutamyl endopeptidase activity, which has previously been isolated from Alcalase™ and was shown to specifically cleave peptide bonds after glutamic and to a lesser extent aspartic acid residues in proteins/peptides.

Whey protein concentrates and isolates containing a minimum of 75% protein may be produced using a variety of manufacturing techniques such as membrane fractionation and concentration (whey protein concentrates (WPC's), isolates (WPI's) and enriched α-lactalbumin and β-lactoglobulin isolates) and ion exchange (whey protein isolates and enriched α-lactalbumin and β-lactoglobulin isolates). Examples of typical high protein products available commercially are WPC 75 (75% protein) and WPI 90 (90% protein). While we have referred to a whey protein hydrolysate, in particular a hydrolysate generated from WPC 75, it is also possible to use substrates such as WPI 90 and whey protein fractions enriched in α-lactalbumin and β-lactoglobulin as protein substrates for hydrolysate manufacture. Total milk or total milk proteins may also be used.

These high protein products may be enzymatically modified using various food grade, proteolytic enzymes to produce a wide range of hydrolysates with low, medium or high degrees of hydrolysis (different levels of breakdown of proteins to their constituent peptides and or amino acids). These hydrolysates may be further modified using membrane or other fractionation techniques to produce products with particular or desirable molecular weight profiles or products enriched in peptides with particular or desirable bioactive properties (FitzGerald and Meisel, 2003).

The general protocol for preparing the active composition(s) comprises: providing milk or milk proteins, or (milk) protein or whey concentrate or isolate; where applicable reconstituting the milk or whey protein concentrate or isolate to form an aqueous protein solution, adding a food grade proteolytic enzyme to the solution, and holding said solution under conditions suitable to effect the desired degree of hydrolysis to produce a whole hydrolysate. A preferred proteolytic enzyme comprises Alcalase™ supplied by Novo Nordisk A/S. Heating the whole hydrolysate inactivates the proteolytic enzyme. The liquid hydrolysate, in one embodiment of the invention, may be fractionated using ultrafiltration membranes to produce a fractionated product capable of endothelial function improvement. The whole and fractionated products are preferably dried and suitably flavoured to render them convenient and acceptable for oral administration in VD/CVD therapy. The products may be reconstituted in, for example, water or milk, before ingestion to improve vascular endothelial function.

The invention will be further illustrated by the following Examples.

### EXAMPLES

A. Preparation of Whey Protein Hydrolysates

Generation of Whey Protein Hydrolysates

Laboratory-scale hydrolysis experiments were initially carried out in a 500 ml reaction vessel containing 8% (w/w) protein. Aqueous solutions of WPC 75 were allowed to hydrate for one hour at room temperature with gentle mixing. The protein solution was then equilibrated at 50° C. for 30 min and the pH adjusted to 7.0 with 1.0 M NaOH before enzyme addition. Alcalase™ 2.4 L (Novo Nordisk A/S (Bagsvaerd, Denmark) was added at an enzyme:substrate (E:S) ratio of 1.5% (w/w), i.e., weight of protein in enzyme preparation/weight of whey protein. The pH was maintained constant at pH 7.0 during hydrolysis using a pH stat (718 Stat Titrino, Metrohm, Herisau, Switzerland). The degree of hydrolysis (DH %), defined as the percentage of peptide bonds cleaved, was calculated from the volume and molarity of NaOH used to maintain constant pH (Adler-Nissen, 1986). On complete hydrolysis, hydrolysate samples were heated at 80° C. for 20 min to inactivate enzyme activity. Larger scale hydrolysis experiments (50-1000 L) were performed in accordance with the above experimental parameters as described below.

Membrane Processing of WPC-Alcalase Hydrolysates

A bench-scale ultrafiltration system (Koch Membrane Systems, Stafford, England) fitted with 5 kDa NMWCO spiral cartridge (S2 HFK-328-VYV, Koch Membrane Systems, Stafford, England) was used. Hydrolysate samples were adjusted to pH 6.2 and were equilibrated at 50° C. Ultrafiltration was carried out at 50° C. to a volume concentration factor (VCF) of between 4.5 and 5.0.

Generation of Whey Protein Hydrolysates at Pilot and Semi-Industrial Scales

The hydrolysis methods were adapted from laboratory bench-scale methods described above but modified to suit the requirements of scaled up pilot and semi-industrial GMP (Good Manufacturing Practice) and HACCP (Hazard Analysis Critical Control Points) controlled processes. The product selected for the human study herein, was a fractionated WPC hydrolysate, i.e. the kDa permeate from an Alcalase™ hydrolysate of WPC.

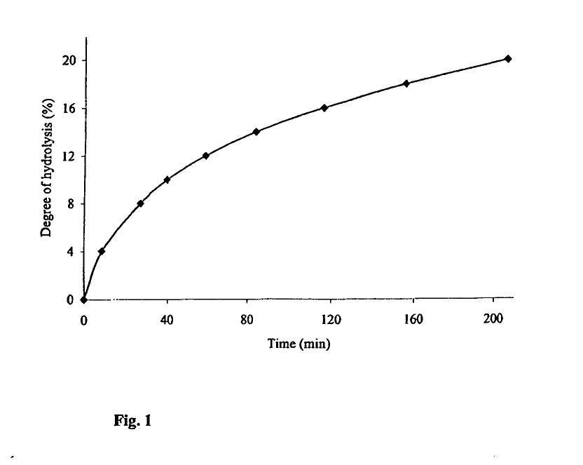

A typical hydrolysis curve for the hydrolysis of WPC 75 with Alcalase™ 2.4 L, a food-grade B. licheniformis proteinase preparation, is shown in FIG. 1. This curve demonstrates that, under the reaction conditions studied, the degree of hydrolysis (DH) plateaus out at ˜19% after ˜200 min incubation at 50° C.

B. Characterisation of Whey Protein Hydrolysate

Gel Permeation HPLC of Whey Protein Hydrolysates

Gel permeation HPLC (GP-HPLC) was performed using a Waters HPLC system, comprising a model 1525 binary pump, a model 717 Plus autosampler and a model 2487 dual λ absorbance detector interfaced with a Breeze™ data-handling package (Waters, Milford, Mass., USA). Hydrolysate samples were diluted to 0.25 g protein equivalent/100 ml in H₂O, filtered through 0.2 μm syringe filters and 20 μl applied to a TSK G2000 SW separating column (600×7.5 mm ID) connected to a TSKGEL SW guard column (75×7.5 mm ID). Separation was by isocratic elution with a mobile phase of 0.1% TFA in 30% acetonitrile, at a flow rate of 1.0 ml min⁻¹. Detector response was monitored at 214 nm. A calibration curve was prepared from the average retention times of standard proteins and peptides (Smyth & FitzGerald, 1997). The void volume (Vₒ) was estimated with thyroglobulin (600 000 Da) and the total column volume (Vₜ) was estimated with L-tyrosine.HCl (218 Da).

Analytical Reversed-Phase (RP) HPLC Analysis of Peptides in WPC Hydrolysates, Retentates and Permeates

RP-HPLC was carried out on the WPC hydrolysates and associated retentates and permeates using a Waters HPLC (Waters, Milford, Mass., U.S.) comprising of a 1525 Binary HPLC pump, 717 Plus Autosampler and 2487 dual wavelength absorbance detector set at 214 and 280 nm. The detector was interfaced with a Waters Breeze™ data handling package (Waters, Milford, Mass., U.S.). The column used was a Phenomenex Jupiter (C18, 250×4.6 mm I.D., 5 μm particle size, 300 Å pore size) separating column (Phenomenex, Cheshire, UK) with a Security Guard™ system containing a C18 (OSD) wide pore cartridge (30×4 mm ID., Phenomenex, Cheshire, UK). The column was equilibrated with solvent A (0.1% TFA in water) at a flow rate of 1 mL/min and peptides were eluted by a linear increase of solvent B (0.1% TFA in 80% acetonitrile, 20% water) from 0% to 100% over 30 min (flow rate 1 ml/min). Detector response was measured at 214 and 280 nm. Hydrolysate samples were diluted to 0.25% (w/v) protein equivalent in deionised/distilled water, filtered through 0.2 μm syringe filters and 20 μL applied to the column.

Simulated Gastrointestinal Digestion

Hydrolysate samples were subjected to a two-stage simulated gastrointestinal digestion (SGID) process. Hydrolysates were diluted to 2.0% (wt/wt) protein and the pH reduced to 2.0 using 1 N HCl. Following pre-incubation (37° C., 30 min), pepsin (E:S, 1:40 wt/wt) was added to 20 ml of gently stirring hydrolysate and the reaction was incubated at 37° C. After 90 min, the pH was adjusted to 7.5 by adding 20 ml of 0.4 M Na₂HPO₄—NaH₂PO₄ buffer pH 7.5. Corolase PP (E:S, 1:100 wt/wt) was then added and the sample was further incubated at 37° C. while stirring. After 150 min the hydrolysate was heated at 80° C. for 20 min to terminate enzyme activity, cooled and then stored at −20° C. Control hydrolysate samples without pepsin and Corolase PP (i.e., non-SGID) were subjected to identical treatments as test samples (Walsh et al., 2004)

The molecular mass distribution profiles of the hydrolysate obtained following hydrolysis of WPC with Alcalase™ at 500 g scale before and after simulated gastrointestinal digestion (SGID) are outlined in Table 2.

TABLE 2

Hydrolysate

| | | | |
|---|---|---|---|
| Mol. mass | WPC intact | −SGID | + SGID |

| | | |
|---|---|---|
| range (kDa) | (%)* | (%) |

| | | | |
|---|---|---|---|
| >10 | 71.32 | 4.56 | 1.77 |
| 5-10 | 10.86 | 2.83 | 1.21 |
| 2-5 | 14.44 | 10.89 | 3.97 |
| 1-2 | 1.91 | 16.06 | 10.56 |
| 0.5-1.0 | 0.43 | 21.54 | 23.87 |
| <0.5 | 1.04 | 44.12 | 58.61 |

*values are areas within a defined molecular mass distribution, expressed as % of total area of a chromatogram at 214 nm.

The results show that significant hydrolysis of intact whey proteins has taken place following incubation with Alcalase™ and that SGID results in further degradation of hydrolysate peptides resulting in increased amounts of peptide material of less than 0.5 kDa. The molecular mass distribution profiles of the WPC Alcalase™ hydrolysate at 50 L scale, and the associated 5 kDa permeates and retentates obtained following ultrafiltration at 50° C. pH 6.2 are shown in Table 3.

TABLE 3

| Mol. mass | hydrolysate | permeate | retentate |
|---|---|---|---|
| range (kDa) | (%)* | | |
| >10 | 0.25 | 0.16 | 2.05 |
| 5-10 | 0.30 | 0.45 | 2.51 |
| 2-5 | 5.59 | 7.84 | 14.98 |
| 1-2 | 14.06 | 15.38 | 20.22 |
| 0.5-1.0 | 25.15 | 22.38 | 20.67 |
| <0.5 | 54.65 | 53.79 | 39.57 |

*values are areas within a defined molecular mass distribution, expressed as % of total area of a chromatogram at 214 nm.

The results again show significant degradation of intact whey proteins and a partitioning of low molecular mass peptides in the 5 kDa permeate fraction. The permeate fraction contained approximately 80% of peptide material<2 kDa. Table 4 summarises the molecular mass distribution profiles obtained following WPC hydrolysis with Alcalase™ at semi-pilot scale (1000 L). The molecular mass distribution profiles for WPC 75 hydrolysates, 5 kDa ultrafiltration permeates and retentates following manufacture at 1000 L are shown.

TABLE 4

| Mol. mass | −SGID | | | +SGID | | |
|---|---|---|---|---|---|---|
| range (kDa) | hydrolysate (%)* | permeate | retentate | hydrolysate (%) | permeate | retentate |
| >10 | 4.47 | 0.13 | 8.73 | 2.46 | 0.30 | 4.98 |
| 5-10 | 3.80 | 0.48 | 7.18 | 2.21 | 0.10 | 4.02 |
| 2-5 | 15.09 | 10.43 | 19.90 | 9.29 | 5.44 | 13.07 |
| 1-2 | 16.99 | 17.81 | 16.42 | 14.59 | 12.82 | 15.84 |
| 0.5-1.0 | 21.49 | 25.72 | 17.39 | 24.84 | 26.85 | 21.70 |
| <0.5 | 38.16 | 45.44 | 30.38 | 46.61 | 54.50 | 40.39 |

*values are areas within a defined molecular mass distribution, expressed as % of total area of a chromatogram at 214 nm

Ultrafiltration through 5 kDa membranes resulted in increased levels of low molecular mass peptides (<2 kDa) in the permeate fraction. In addition SGID resulted in further degradation of peptides in the hydrolysate, permeate and retentate fractions, yielding increased levels of low molecular mass peptides.

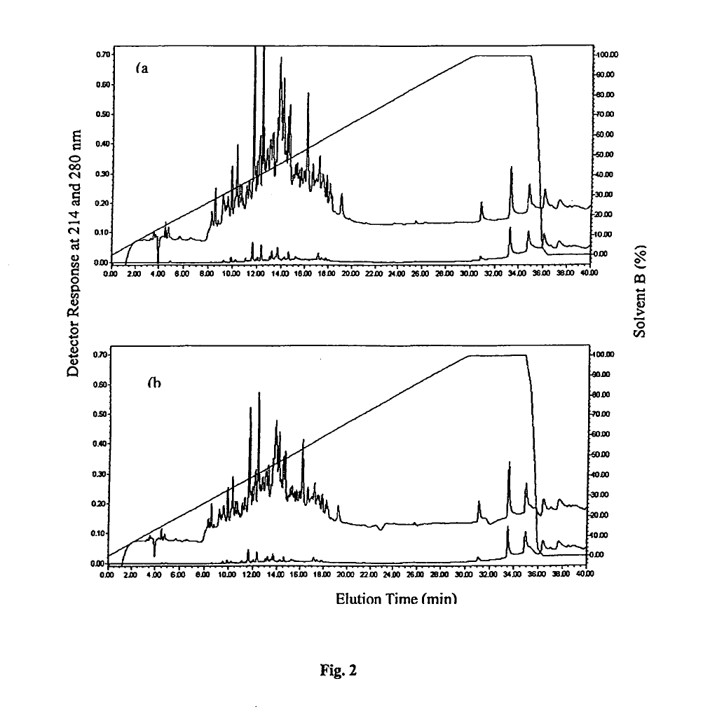

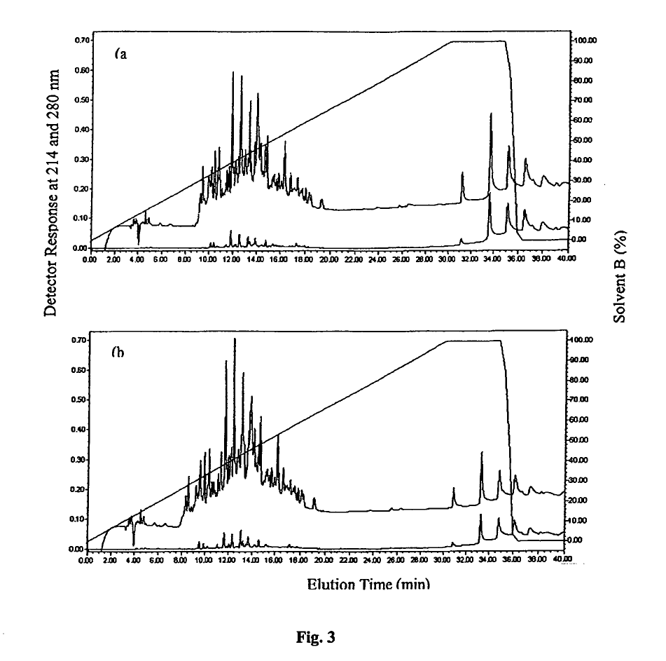

FIG. 2 shows the reversed-phase HPLC profiles obtained for WPC-Alcalase™ hydrolysate manufactured at 1000 L (a) with and (b) without SGID. No major changes in the RP-HPLC profiles were observed after SGID treatment. FIG. 3 shows the RP-HPLC profiles of the 5 kDa permeate (a) before and (b) after SGID. Again no major changes in the RP profiles were evident for the 5 kDa permeate samples after SGID treatment.

Amino Acid Composition of the WPC 75 Hydrolysate

The amino acid composition of the WPC 75 hydrolysate 5 kDa permeate fraction (semi-pilot scale) obtained following ultrafiltration through a 5 kDa molecular mass cut-off membrane compared to unhydrolysed WPC 75 is shown in Table 5. The amino acid profile of the permeate fraction is very similar to that of WPC.

TABLE 5

| | WPC hydrolysate | |
|---|---|---|
| | 5 kDa permeate | Unhydrolysed WPC 75 |
| Amino Acid | (g/100 g powder) | (g/100 g powder) |
| Trp | 0.98 | 1.5 |
| Asp | 6.58 | 8.55 |
| Ser | 4.14 | 4.12 |
| Glu | 13.80 | 14.3 |
| Gly | 2.22 | 1.5 |
| His | 0.77 | 1.42 |
| Arg | 1.76 | 2.00 |
| Thr | 5.66 | 5.40 |
| Ala | 4.04 | 3.80 |
| Pro | 4.55 | 4.87 |
| Cys | 1.38 | 2.02 |
| Tyr | 2.59 | 2.25 |
| Val | 4.08 | 4.65 |
| Met | 1.57 | 1.57 |
| Lys | 6.18 | 7.12 |
| Ile | 3.92 | 4.80 |
| Leu | 8.12 | 8.4 |
| Phe | 2.61 | 2.32 |

C. In Vivo Examples—Effect of Whey Protein Hydrolysate on Endothelial Function

The effect of ingesting a fractionated whey protein hydrolysate on endothelial function was examined in pre-hypertensive human volunteers.

All the participants in the studies described hereunder gave written informed consent to participate. The studies had prior approval by the local Committee on Medical Research Ethics. Exclusion criteria were history of atopy (asthma, eczema or hay-fever), allergy to cow's milk, lactose intolerance, epilepsy and any serious illness that would preclude inclusion.

Fourteen pre-hypertensive volunteers were used to compare 4 weeks of 14 g per day of fractionated whey protein hydrolysate (5 kDa permeate manufactured as described above using WPC75 as a substrate) with placebo (unhydrolysed WPC 75) in a randomised, placebo-controlled double blind, crossover trial with a one-week washout period between phases. Serum ACE, plasma renin activity, Angiotensin II, aldosterone levels and blood pressure were measured at weekly intervals. Each subject attended for vascular studies, which were performed at the end of each treatment phase, as detailed below. Serum cholesterol was measured after 4 weeks at the end of each arm.

Serum ACE Inhibitory Activity Measurements

Serum ACE inhibitory activity was assayed spectrophotometrically. The method is based on the liberation of furylacryloylphenylalanine (FAP) from the substrate N-[3-(2-furyl)acryloyl]-L-phenylalanylglycylglycine (FAPGG) catalysed by ACE. Hydrolysis of FAPGG results in a decrease in absorbance at 340 nm.

Plasma Renin

The plasma renin activity assays were performed by an in-house radioimmunoassay using a standard kit (DiaSorin, Saluggia, Italy). The intraassay and interassay coefficients of variability were both 12%.

Plasma Angiotensin II

Blood samples were taken into chilled tubes containing ethylene diamine tetracetic acid, enalkiren (a renin inhibitor), enalapril (an in vitro ACE inhibitor) and O-phenanthroline. After extraction of the plasma samples, angiotensin II was assayed by a competitive radioimmunoassay (Euro-Diagnostica, Netherlands). The radioimmunoassay uses a rabbit anti-angiotensin II antiserum and radio-iodinated angiotensin II tracer.

Aldosterone Assays

Aldosterone assays were performed by an in-house radioimmunoassay using a standard commercial kit (Sorin Biomedica, Saluggia, Italy). The intraassay and interassay coefficients of variability were <9% for both.

Blood Pressure Measurements

Blood pressure was measured on the left arm with a DINAMAP™ PRO 100 monitor (Criticin, Berkshire, England) with subjects in the semi-recumbent position for 30 minutes. Three consecutive blood pressure measurements were recorded and the mean used in the statistical analysis.

Vascular Studies

After an overnight fast, patients attended a temperature-controlled laboratory (24° C.±0.5° C.) at 8 am. On each study day, the subject lay supine, and a mercury-in-silastic strain gauge (Medasonics) was applied to each forearm at the point of maximal muscle bulk. The position of the gauge was determined by measuring the distance from the olecranon process and was kept constant for each individual between study days. Cuffs were placed around each wrist and upper arm and were attached to a rapid cuff inflator (Hokanson). Forearm Blood Flow (FBF) measurements were taken from both arms over a 2-minute period at the end of each dose interval, during which the wrist cuffs were inflated to 200 mm Hg to exclude the hand circulation. Each measurement was taken as the mean of five readings, which were obtained during periodic inflation of the upper arm cuffs to 40 mm Hg (to occlude venous outflow) for 10 seconds in every 15 seconds. Data from the strain gauges were processed by a plethysmograph (Medasonics) and analyzed using PC computer hardware and Powerlab Chart 5 software by AD Instruments (Oxfordshire, UK). Heart rate and blood pressure were measured by a semi-automated sphygmomanometer (Dinamap) after each infusion.

A 27-gauge needle was inserted into the brachial artery of the non-dominant arm under local anaesthesia, and 0.9% saline was infused for at least 30 minutes prior to infusion of acetylcholine. Strain gauge measurements were taken at 10-minute intervals until stable readings, defined by three consecutive measurements with less than 10% variability, were obtained. The mean of the ratio of measurements from both arms at these three time points was taken as the baseline ratio of forearm blood flow. Drugs were then infused (see below) into the study arm with a constant rate infuser. FBFs were measured at each baseline and during the last two minutes of each drug infusion.

Drug Infusions

Acetylcholine (Ach) was infused at doses of 50 and 100 nmol/min for 5 minutes each and then sodium nitroprusside (SNP) at a dose of 37.8 nmol/min was infused for 5 minutes. Between the different drugs, the drug infusion set was flushed with saline for 20 to 30 minutes to allow sufficient time for the FBF to return to baseline. Acetylcholine acts on the endothelium as a potent endothelial dependent vasodilator and sodium nitroprusside acts as an endothelial independent vasodilator.

Statistical Analysis

Forearm blood flow (FBF) (expressed as mL·Min⁻¹ per 100 mL forearm volume) were measured by plethysmography in both arms. They were converted into the ratio between the FBF in the infused arm and the FBF in the control arm and then expressed as percentage change in FBF ratio from baseline. FBF measurements for individual subjects were compared between treatments by two-way analysis of variance with replication. A value of p<0.05 was considered significant and a value of p<0.01 highly significant. This statistical methodology has been validated as being most accurate in reflecting true differences in blood flow characteristics. The plethysmography technique itself is well suited to relatively small studies in adults, being able to detect a change of <20% with >90% power and p<0.05 in studies of ˜20 individuals studied on separate occasions. Clinical characteristics between study visits were compared using Student's paired t tests.

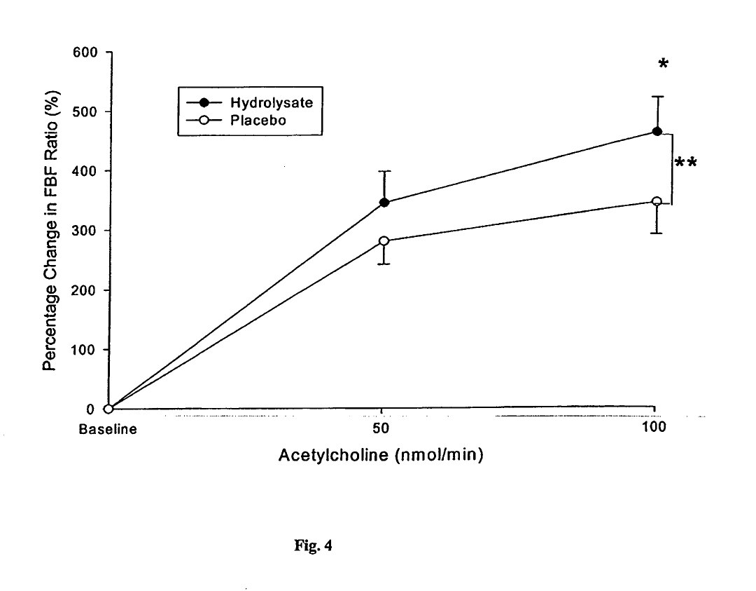

A significant increase in vasodilatation was however seen between the hydrolysate (WPC75 Alcalase™ 5 kDa permeate) and placebo (unhydrolysed WPC 75) with regard to Acetylcholine infusion (P=0.002) during forearm venous occlusion plethysmography implying a substantial improvement in endothelial function (FIG. 4). At the higher dose of acetylcholine tested, the improvement in endothelial dependent vasodilatation was 34%. At the lower test dose of acetylcholine, the improvement in endothelial dependent vasodilatation was 24%. In contrast, the hydrolysed WPC75 had no effect on the endothelial independent vasodilator sodium nitroprusside responses compared to placebo. The fact that acetylcholine responses increased while sodium nitroprusside responses remained unchanged is typical of a treatment that improves endothelial dependent vasodilatation.

This large improvement in endothelial function may mean that vascular or vascular-related events like myocardial infarctions, coronary artery disease, and strokes should also be reduced. This could apply to both people who have not yet had a myocardial infarction or stroke as well as to those who have already survived a myocardial infarction or stroke. In that sense, the hydrolysate of the present invention could be both preventative and therapeutic.

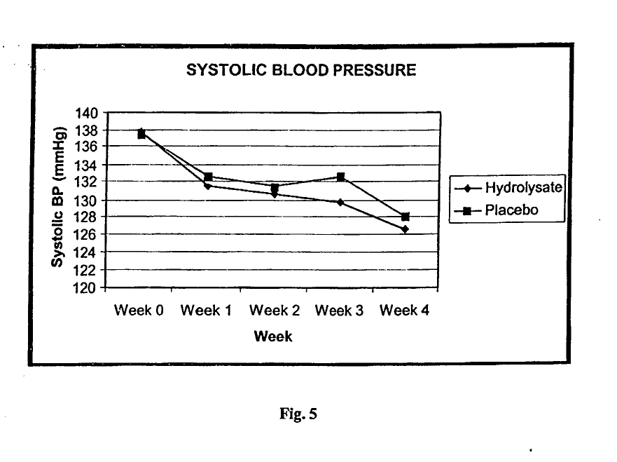

Table 6 shows the effect of the hydrolysate on renin, ACE, AII, Aldosterone, cholesterol and systolic BP in comparison to a placebo. FIG. 5 shows changes in systolic blood pressure over 4 weeks ingestion of the test hydrolysate sample in comparison to the placebo.

TABLE 6

| | | 1 week | 2 weeks | 4 weeks |
|---|---|---|---|---|
| Systolic BP | Placebo | 132 ± 4 | 131 ± 4 | 128 ± 4 |
| | Hydrolysate | 131 ± 4 | 130 ± 4 | 127 ± 4 |
| Renin | Placebo | 0.4 ± 0.1 | 0.4 ± 0.1 | 0.3 ± 0.1 |
| | Hydrolysate | 0.4 ± 0.1 | 0.3 ± 0.1 | 0.3 ± 0.1 |
| ACE | Placebo | 29 ± 3 | 29 ± 3 | 26 ± 3 |
| | Hydrolysate | 28 ± 3 | 28 ± 3 | 28 ± 3 |
| AII | Placebo | 16 ± 1 | 16 ± 1 | 15 ± 1 |
| | Hydrolysate | 15 ± 1 | 15 ± 1 | 15 ± 1 |
| Aldosterone | Placebo | 66 ± 14 | 58 ± 11 | 50 ± 7 |
| | Hydrolysate | 47 ± 7 | 49 ± 9 | 50 ± 7 |
| Cholesterol | Placebo | | | 5.4 ± 0.2 |
| | Hydrolysate | | | 5.4 ± 0.2 |

At baseline, systolic BP was 138±4, cholesterol 5.7±0.2, renin 0.6±0.2, ACE 31±4, AII 16±1, Aldosterone 56±8.

Units are systolic BP—mmHg, Renin—ng/ml/hr, ACE—Iu/ml, AII—pmol/l, Aldosterone—pg/ml

The results indicate that the milk or whey protein hydrolysate of the invention improves endothelial function by some novel mechanism and not by changing the traditional risk factors of cholesterol or BP or ACE. Surprisingly however, the hydrolysate preparation was found to be beneficial on endothelial function without altering ACE activity and without altering blood pressure (Table 6, FIG. 5).

D. Functional Properties of Whey Protein Hydrolysates

Determination of the Nitrogen Solubility Indices of Selected Hydrolysate Samples

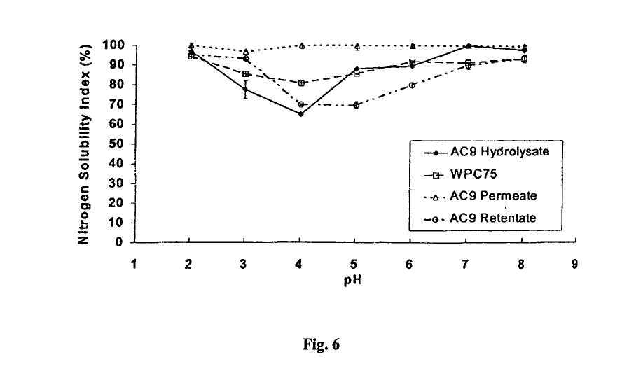

The solubility of 1% (w/w) dispersions of GMP produced WPC 75-Alcalase™ hydrolysate, WPC 75-Alcalase™ 5 kDa permeate and retentate were determined in duplicate between pH 2.0 to pH 8.0. Protein solutions (0.3 g protein) were weighed into pre-weighed 50 ml plastic bottles. Twenty-five grams of distilled deionised water was added. Samples were stirred on an orbital stirrer (Gerhardt Schuttelmaschine RO 10, Bonn, Germany), at speed-setting 5 for 1 h. The dispersions were allowed to remain undisturbed for at least one hour after mixing to allow for hydration of the dispersed protein. The pH of each sample was adjusted to a pH value between pH 2.0 and 8.0 while stirring using 0.1 M NaOH or 0.1 M HCl and water was added to adjust the final weight to 30 g. Samples were left to stand for one hour. Protein samples were then mixed using a magnetic stirrer and an aliquot (9 mL) was removed in duplicate for estimation of total nitrogen content by macro-Kjeldahl. The remaining solutions were centrifuged at 1300×g for 30 min using a Sorvall RC 5C Plus Centrifuge (Sorvall Products, Newtown, Conn., USA). The supernatant was decanted from the pellet and filtered through Whatman No. 1 filter paper (Whatman International, Maidstone, England). Soluble nitrogen in the supernatant was determined in duplicate using the macro-Kjeldahl procedure. Solubility was expressed as the percentage nitrogen content of supernatant divided by the overall nitrogen content in the starting solution. (FIG. 6)

FIG. 6 shows the solubility properties of WPC-Alcalase™ hydrolysate and its associated 5 kDa permeates and retentates manufactured at semi-pilot scale. The results show that the WPC-Alcalase™ 5 kDa permeate has excellent solubility across the entire pH range tested. On the other hand, WPC, WPC Alcalase™ whole hydrolysate and the 5 kDa hydrolysate retentate displayed some reduction (20-30%) in solubility between pH 3.0 and 6.0.

Whipping and Foaming Properties of Selected Hydrolysate Samples

The sample hydrolysates, retentates and permeates (250 mL) were removed from the refrigerator and placed in a water bath at 37° C. for 1 hour with occasional mixing in order to allow any insoluble protein back into solution. Samples were adjusted to pH 2, 4, 6 and 8 using 1N HCl and/or 1N NaOH prior to diluting to 0.5% protein using distilled deionised water. The diluted sample (200 mL) equilibrated at room temperature (20° C.) was then mixed at maximum speed for 10 minutes in a household food mixer (Kenwood Chef Classic KM 400/410, Kenwood Ltd., Hampshire, U.K.). The resulting foam was transferred to a pre-weighed cylindrical polypropylene funnel (104 mm dia., 58.5 mm ht.), which had an internal fused wire mesh base (2 mm). Large bubbles were removed from the foam and the surface of the foam was scrapped flat. The funnel and foam were weighed immediately (To) and weighed again after standing for 15 and 30 min in a graduated cylinder. The procedure was carried out in duplicate for each of the samples at each pH. Percentage foam expansion was calculated from equation (1) and percentage foam stability was calculated from equation (2) at 15 and 30 min.

$$\begin{matrix}Foam Expansion ( \% ) = \frac{Volume of cylinder - Mass of foam in cylinder}{Volume of foam in cylinder} \times 100 & ( 1 ) \\ Foam Stability ( \% ) = \frac{Mass of foam at time ( t )}{Mass of foam at time ( 0 )} \times 100 & ( 2 )\end{matrix}$$

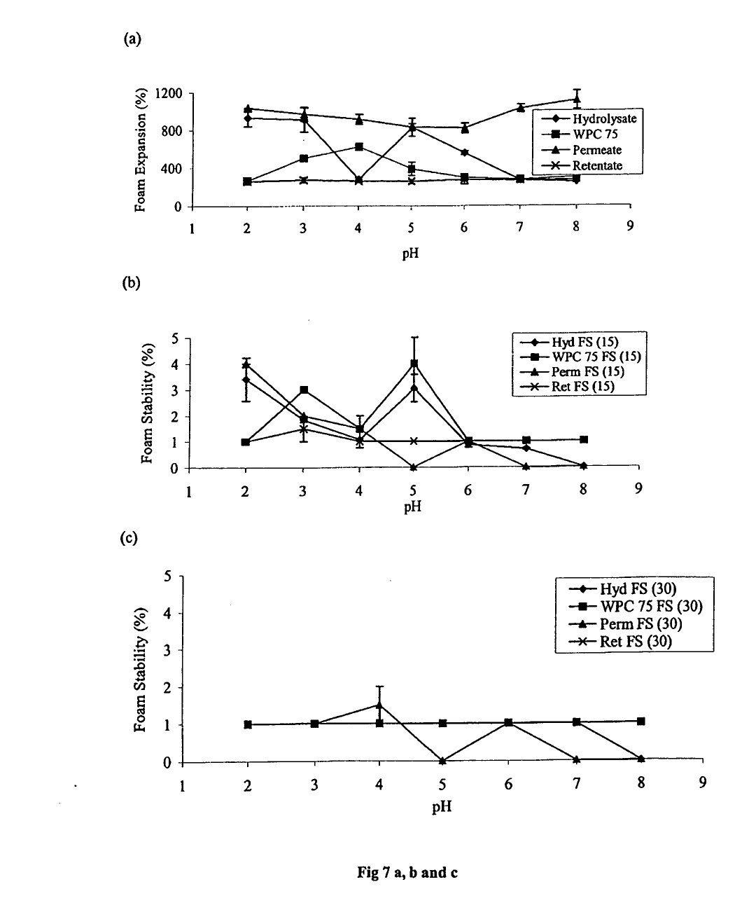

FIG. 7 shows the foam expansion (FE) and foam stability (FS) properties of WPC hydrolysate and associated 5 kDa permeate and retentate fractions as a function of pH. The 5 kDa permeate fraction had high FE values (>800%) across the pH range tested while the corresponding retentate fraction displayed very low FE between pH 2.0-8.0 (FIG. 7a). FS was very low (<5%) over the pH range for all test samples 15 min after foam formation (FIG. 7b). FS decreased further on standing for 30 min (FIG. 7c). This low foam stability may be desirable in beverage products for human consumption.

It is anticipated that the protein hydrolysate products resulting from the application of the invention could, for example, be incorporated into functional foods and nutraceutical products such as ready to drink or mix beverages, nutritional bars and dietary supplements or pharmaceutical products (in the form of tablets, capsules, tinctures or creams) where therapeutically or prophylactically effective amounts could be administered either orally, topically or systemically. In any of these formats, the products of the invention could be used to improve an individual's vascular health with the expectation that it may help to considerably reduce the chance of at risk individuals having myocardial infarctions or strokes.

The milk protein hydrolysate products of the invention may be used in the treatment, prevention or beneficial management of vascular conditions and diseases, including cardiovascular diseases and disorders.

Pharmaceutically acceptable carriers and adjuvants well known in the art may be included in the compositions comprising the hydrolysate products. The compositions may also include a drug entity. They may be manufactured in the form of an injectable, solid dose or liquid dose form, including tablets, capsules and the like, for oral, systemic or topical administration.

A composition comprising a milk protein hydrolysate of the invention may be effective in preventing, treating and/or alleviating vascular conditions or diseases and/or for treating vascular conditions which are known as risk factors for these, including pre-hypertension, hypertension, hypercholesterolemia and diabetes mellitus. It is also useful for administration to individuals who practice habits, such as smoking, which are known risk factors for the development of vascular conditions or diseases.

The invention is not limited to the embodiments hereinbefore described, with reference to the accompanying drawings, which may be varied in construction and detail.

Appendix

Adler-Nissen, J. (1986). A review of food protein hydrolysis-specific areas. In Enzymatic Hydrolysis of Food Proteins. Elsevier Applied Science, New York, 57-109.

Anderson, T. J. (2003). Nitric oxide, atherosclerosis and the clinical relevance of endothelial dysfunction. Heart Failure Reviews 8(1): 71-86.

Cai, H., Griendling, K. K., and Harrison, D. G. (2003). The vascular NAD(P)H oxidases as therapeutic targets in cardiovascular diseases. Trends in Pharmacological Science 24(9): 471-478.

Cohn J. N., Quyyumi A. A., Hullenberg N., Jamerson K. A. (2004). Surrogate markers for cardiovascular disease functional markers. Circulation, 109 (supplement IV) IV-31 to IV-46.

Cooper, D., Stokes, K. Y., Tailor, A. and Granger, D. N. (2002). Oxidative stress promotes blood cell-endothelial cell interactions in the microcirculation. Cardiovascular Toxicology. 2(3): 165-180.

Farre A L, and Casado S, (2001). Heart failure, redox alterations, and endothelial dysfunction. Hypertension, December 1; 38 (6):1400-5.

Farquharson C and Struthers A D, (2000). Spironolactone increases nitric oxide bioactivity, improves endothelial vasodilator function and suppresses vascular AI/AII conversion in patients with chronic heart failure. Circulation 2000, 101, 594-597.

Ferrario C M., Strawn W. (2002). The hypertension-lipid connection: insights into the relation between angiotensin II and cholesterol in atherogenesis. Amer. J. Med. Sciences, 323:17-24.

FitzGerald, R. J., Murray, B., and Walsh, D. J. (2004). Hypotensive peptides from milk proteins. J. Nut. 134: 980S-988S

FitzGerald and Meisel, (2003). Milk protein hydrolysis and bioactive peptides. In: Advanced Dairy Chemistry, Third Edition, Part B, Chapter 14 (eds. Fox, P. F. and McSweeney, P.), Kluwer Academic/Plenum Publishers, New York, pp. 675-698.

Gokce N., Keaney J. F., Hunter L. M. et al. (2003). Predictive value of non-invasively determined endothelial dysfunction for long term cardiovascular events in patients with peripheral vascular disease. J Am Coll Cardio, 41: 1769-1775.

Heitzer T., Schlinzig T., Krohn K., Neinertz T., Munzel T. (2001). Endothelial dysfunction, oxidative stress and risk of cardiovascular events in patients with coronary artery disease. Circulation, 104: 2673-2678.

Modena M. G., Bonetti L, Coppi F., Bursi F., Rossi R. (2002). Prognostic role of reversible endothelial dysfunction in hypertensive postmenopausal women. J Am Coll Cardiol; 40: 505-510.

Murray, B. A., Walsh, D. J., and FitzGerald, R. J. (2004). Modification of the furanacryloyl-L-phenylalanylglycylglycine assay for determination of angiotensin converting enzyme inhibitory activity. Journal of Biochemical and Biophysical Methods, 59: 127-137.

O'Driscoll G., Green D., Rankin J Stanton K., Taylor T. (1997). Improvement in endothelial dysfunction by ACE inhibition in insulin dependent diabetes mellitus. J Clin Invest, 100: 678-684.

Olsen et al., (2001). Endothelial dysfunction in resistance arteries is related to high blood pressure and circulating low density lipoproteins in previously treated hypertension. Amer. J. Hypertension, 14:861-867.

Perticone F., Ceravolo R., Pujia A. et al. (2001). Prognostic significance of endothelial dysfunction in hypertensive patients. Circulation, 104: 191-196.

Schachinger V., Britten M. B., Zeiher A. M. (2000). Prognostic impact of coronary vasodilator dysfunction in adverse long-term outcome of coronary heart disease. Circulation, 101: 1899-1906.

Smyth, M. & FitzGerald, R. J. (1997). Characterisation of a new chromatography matrix for peptide molecular mass determination. International Dairy Journal 7 571-577.

Spellman, D., Kenny, P., O'Cuinn, G. and FitzGerald, R. J. (2005), Aggregation properties of whey protein hydrolysates generated with Bacillus licheniformis proteinase activities. J. Agric. Food Chem., 53: 1258-1265.

Suwaidi et al., (2000). Long-term follow-up of patients with mild coronary artery disease and endothelial dysfunction Circulation, 101:948-954.

Targonski P. V., Bonetti P. O., Pumper G. M. et al. (2003). Coronary endothelial function is associated with increased risk of cerebrovascular events. Circulation, 107: 2805-2809.

Vita J A, Keaney J. F. (2002) Endothelial function. A barometer for cardiovascular risk. Circulation, 106: 640-642.

Vogel R. A. (2003). Heads and hearts. The Endothelial Connection. Circulation, 107: 2766-2768.

Walsh, D. J., Bernard, H., Murray, B. A., MacDonald, J., Pentzien, A.-K., Wright, G. A., Wal, J-M., Struthers, A., Meisel, H. and FitzGerald, R. J. (2004). In Vitro Generation and Stability of the Lactokinin β-Lactoglobulin f(142-148). J. Dairy Sci. 87: 3845-3857.

Widlansky M. E., Gokee N., Keaney J. F., Vita J. A. (2003). The clinical implications of endothelial dysfunction. J Am Coll Cardiol, 42, 1149-1160.

1. A composition for use in the treatment or prophylaxis of conditions mediated by endothelial function in mammals, comprising a milk protein hydrolysate prepared by treating milk or milk whey protein with a proteolytic enzyme having subtilisin or subtilisin-like activity and/or glutamyl endopeptidase or glutamyl endopeptidase-like activity.

2. The composition of claim 1 wherein the proteolytic enzyme is derived from Bacillus species.

3. The composition of claim 1 wherein the proteolytic enzyme is derived from Bacillus licheniformis.

4. The composition of claim 1 wherein the proteolytic enzyme comprises Alcalase™ from Bacillus licheniformis.

5. The composition of claim 1 wherein the hydrolysate comprises a fractionated whey protein hydrolysate.

6. The composition of claim 5 wherein the whey fraction comprises a molecular weight fraction of 5 kDa or less.

7. The composition of claim 6 wherein the whey fraction contains greater than 60% of peptide material having a molecular weight of less than 2 kDa.

8. The composition of claim 7 wherein the whey fraction comprises greater than 70% of peptide material having a molecular weight of less than 2 kDa.

9. The composition of claim 8 wherein the whey fraction comprises greater than 80% of peptide material having a molecular weight of less than 2 kDa.

10. The composition of claim 9 wherein the whey fraction comprises greater than 55% of peptide material having a molecular weight of less than 1 kDa.

11. The composition of claim 10 wherein the whey fraction comprises greater than 65% of peptide material having a molecular weight of less than 1 kDa.

12. The composition of claim 11 wherein the whey fraction comprises greater than 40% of peptide material having a molecular weight of less than 500 Daltons.

13. The composition of claim 1 wherein the milk protein hydrolysate has a degree of hydrolysis of greater than 10%, preferably from about 15% to about 25%.

14. The composition of claim 13 wherein the degree of hydrolysis is approximately 19%.

15. The composition as claimed in claim 1 comprising between 5 g and 18 g of the milk protein hydrolysate.

16. The composition of claim 15 comprising about 14 g of the milk protein hydrolysate.

17. The composition of claim 1 wherein the milk protein hydrolysate has greater than 80% solubility between pH 2.0 to 8.0.

18. The composition of claim 17 wherein the milk protein hydrolysate has greater than 95% solubility between pH 2.0 to 8.0.

19. The composition of claim 1 wherein the whey protein hydrolysate has a foam stability of less than 10% after 15 minutes standing following foam formation.

20. The composition of claim 19 having a foam stability of less than 5% after 15 minutes standing following foam formation.

21. The composition of claim 1 in which the whey protein hydrolysate has an amino acid composition as outlined for the permeate fraction in the examples presented herein.

22. The composition of claim 1 for use in the treatment, prophylaxis or management in mammals of vascular diseases, disorders or conditions including coronary artery disease, cerebral vascular disease or peripheral vascular disease, or of disorders which are risk factors for the development of such vascular diseases, disorders or conditions including coronary artery disease, pre-hypertension, hypertension, hypercholesterolemia or diabetes mellitus.

23. The composition of claim 1 for diagnosis, prophylaxis, treatment or beneficial management of a mammalian vascular or cardiovascular risk state disease, disorder, or condition which is affected by endothelial dysfunction.

24. The composition of claim 23 wherein the mammalian disease, disorder, or condition comprises vascular diseases or disorders selected from coronary artery disease, cerebrovascular diseases, stroke, and peripheral arterial disease and risk states associated with such diseases or disorders including pre-hypertension, hypertension, hypercholesterolemia and diabetes mellitus.

25. A process for the preparation of a milk protein hydrolysate especially for use in stimulating endothelial function comprising the steps of:
optionally reconstituting or hydrating a milk protein;
hydrolysing a milk protein with a food grade proteolytic enzyme having subtilisin or subtilisin-like and/or glutamyl endopeptidase or glutamyl endopeptidase-like activity; and
fractionating the hydrolysed milk protein product.

26. The process of claim 25 wherein the proteolytic enzyme is derived from Bacillus species.

27. The process of claim 25 wherein the proteolytic enzyme is derived from Bacillus licheniformis.

28. The process of claim 25 wherein the proteolytic enzyme comprises Alcalase™ from Bacillus licheniformis.

29. The process of claim 25, including pre-treating milk to separate a fraction comprising whey protein and optionally concentrating the whey protein fraction.

30. The process of claim 25 including treating the hydrolysate to separate from it a fraction comprising species of 5 kDa and lower.

31. The process of claim 25, in which the hydrolysis is carried out at a temperature of between 30° C. and 70° C., preferably between 40° C. and 60° C. and most preferably at a temperature around 50° C.

32. The process of claim 25 in which the hydrolysis is carried out at a pH of between 4 and 9, preferably between 6 and 8 and most preferably at around neutral pH.

33. A method for preventing or treating vascular or cardiovascular conditions, disorders or diseases in mammals comprising administering an effective dose of a composition as claimed in claim 1.

34. A method as claimed in claim 33 for treating coronary artery disease, cerebral vascular disease including stroke or peripheral arterial disease.

35. A method as claimed in claim 33 for treating conditions which represent risk factors for vascular disease, including pre-hypertension, hypertension, hypercholesterolemia and diabetes mellitus.

36. A method for monitoring cardiovascular therapy comprising the determination of vascular endothelial function before and after administration of the composition as claimed in claim 1.

37. A dosage form comprising a composition as claimed in claim 1 and one or more suitable carriers and/or adjutants.

38. The dosage form as claimed in claim 37 comprising a delivery system for providing a desirable daily dose.

39. A dosage form as claimed in claim 37 adapted for oral administration.

40. Use of a composition as claimed in claim 1 in the manufacture of a preparation for beneficially modifying endothelial function.

41. Use as claimed in claim 40, wherein the endothelial function is endothelial dependent relaxation activity.

42. Use as claimed in claim 41 wherein the endothelial dependent relaxation activity is endothelial dependent vasodilatation.

43. Use as claimed in claim 41 wherein the endothelial dependent relaxation activity is stimulated by greater than 10%.

44. Use as claimed in claim 41 wherein the endothelial dependent relaxation activity is stimulated by greater than 20%.

45. Use as claimed in claim 41 wherein the endothelial dependent relaxation activity is stimulated by greater than 30%.
